(19) United States
(12) Patent Application Publication
(10) Pub. No.: US 2024/0382476 A1
(43) Pub. Date: Nov. 21, 2024

(54) ANDROGEN RECEPTOR BIOMARKERS FOR CANCER THERAPY

(71) Applicants: Xiang LI, Beijing (CN); Chun JIANG, Hillsborough, CA (US); Yiyou CHEN, San Jose, CA (US)

(72) Inventors: Xiang LI, Beijing (CN); Chun JIANG, Hillsborough, CA (US); Yiyou CHEN, San Jose, CA (US)

(21) Appl. No.: 18/692,204

(22) PCT Filed: Sep. 15, 2022
(86) PCT No.: PCT/US2022/076460

§ 371 Date: Mar. 14, 2024

(30) Foreign Application Priority Data
Sep. 16, 2021 (WO) PCT/CN2021/118866

(51) Int. Cl. A61K 31/497; A61K 31/337; A61K 31/555; A61K 31/7048; A61K 33/243; A61P 35/00; C12Q 1/6886

(52) U.S. Cl. CPC A61K 31/497; A61K 31/337; A61K 31/555; A61K 31/7048; A61K 33/243; A61P 35/00; C12Q 1/6886; C12Q 2600/106; C12Q 2600/158

(57) ABSTRACT

Provided is the use of Androgen Receptor-Low/Negative (AR^{low/-}) status as a biomarker for the efficacy of YM155 monobromide in cancer therapy, and related kits, compositions, and methods for diagnosing and treating cancer in a subject in need thereof.

### CROSS-REFERENCE TO RELATED APPLICATIONS

This application claims priority to PCT/CN2021/118866, filed Sep. 16, 2021, which is incorporated by reference in its entirety.

### BACKGROUND

### Technical Field

Embodiments of the present disclosure relate to the use of Androgen Receptor-Low/Negative (AR^{low/-}) status as a biomarker for the efficacy of YM155 monobromide in cancer therapy, and related kits, compositions, and methods for diagnosing and treating cancer in a subject in need thereof.

### Description of the Related Art

YM155 monobromide is a small-molecule that exhibits potent antitumor activity (see, e.g., Minematsu et al., Drug Metabolism and Disposition, 37:619-628, 2008). YM-155 exerts anti-tumor effects in various in vivo cancer models, including prostate, pancreatic, and lung cancer (see, e.g., Nakahara et al., Cancer Research 67:8014-8021, 2007; and Na et al., PLOS One 7 (6), 2012).

However, there is a need in the art to better predict the anti-cancer therapeutic efficacy of YM155 monobromide, and thereby identify patients that will benefit most from treatment with this chemotherapeutic, and others.

### BRIEF SUMMARY

Embodiments of the present disclosure include methods for treating an Androgen Receptor-Low/Negative (AR^{low/-}) prostate cancer in a subject in need thereof, comprising
administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof,
thereby treating the AR^{low/-} prostate cancer in the subject in need thereof.

Certain embodiments comprise:
(a) determining (i) AR expression levels, (ii) AR mutation status, and/or (iii) the presence or absence of a neuroendocrine prostate cancer (NEPC) or a small cell carcinoma (SmCC), in a tissue sample from the subject; and
(b) administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, to the subject if (i) AR expression levels in the tissue sample are absent (undetectable) or decreased relative to a control or reference, (ii) if the tissue sample lacks an activating AR mutation relative to wild-type AR, and/or (iii) if an NEPC or an SmCC is present in the sample.

Also included are methods for predicting therapeutic response to YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, in a subject with prostate cancer, comprising
(a) determining (i) AR expression levels, (ii) AR mutation status, and/or (iii) the presence or absence of a neuroendocrine prostate cancer (NEPC) or a small cell carcinoma (SmCC), in a tissue sample from the subject; and
(b) (i) characterizing the subject as responsive to YM155 monobromide therapy if (i) AR expression levels in the tissue sample are absent (undetectable) or decreased relative to a control or reference, (ii) if the tissue sample lacks an activating AR mutation relative to wild-type AR, and/or (iii) if an NEPC or SmCC is present in the tissue sample; or
   (ii) characterizing the subject as non-responsive to YM155 monobromide therapy if (i) AR expression levels in the tissue sample are detectable or normal relative to a control or reference. (ii) if the tissue sample comprises an activating AR mutation, and/or (iii) if an NEPC and an SmCC are absent in the tissue sample.
thereby predicting therapeutic response to YM155 monobromide in the subject with prostate cancer.

Some embodiments comprise administering the YM155 monobromide to the subject if the subject is characterized as responsive to YM155 monobromide therapy.

Some embodiments comprise determining AR expression levels in the tissue sample by immunohistochemistry (IHC) optionally chromogenic or fluorescent IHC, enzyme linked immunosorbent assay (ELISA), or Western blot on a human AR protein or gene, or by measuring AR mRNA expression levels. Some embodiments comprise administering the YM155 to the subject if AR levels in the tissue sample are undetectable or decreased by about or at least about 2, 3, 4, 5, 6, 7, 8, 9, or 10, 50, or 100-fold or more relative to the AR levels of the control or reference, optionally wherein the control is a healthy tissue.

Certain embodiments comprise determining AR mutation status in the tissue sample by DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene.

In some embodiments, the activating AR mutation is selected from one or more of one or more of AR gene amplification, one or more activating mutations in the ligand binding domain of AR (optionally selected from H874Y, T877A, T877S, T878A, and F876L), an AR splice variant lacking the ligand binding domain of AR (optionally ARV7 or ARV567), and an E3 ligase MDM2 loss-of-function mutation.

In some embodiments, the AR^{low/-} prostate cancer is a castration-resistant prostate cancer (CRPC). In some embodiments, the prostate cancer or CRPC comprises an NEPC, an SmCC, a double negative prostate cancer (DNPC), or an AR low prostate cancer (ARLPC).

In some embodiments. (iii) comprises determining the presence of the NEPC or SmCC in the tissue sample by cell morphology/histology, the absence of AR expression, and/or by immunohistochemistry (IHC), optionally via one or more markers selected from synaptophysin, chromogranin A (CgA), neuron-specific enolase (NSE), and CD56.

Certain embodiments comprise obtaining the tissue sample from the subject. In some embodiments, the tissue sample is a liquid biopsy optionally a blood sample, a surgical sample, or other biopsy sample obtained from the subject, optionally a biopsy of prostate cancer tissue.

In some embodiments, the subject is a human subject, for example, wherein the human subject has received at least 1 or 2 lines of systemic therapy for the prostate cancer and has relapsed from the last systemic therapy, including any one or more of hormonal therapy via surgical or chemical castration (LHRH agonist), chemotherapy, and/or radiopharmaceutical therapy.

Some embodiments comprise administering the YM155 in combination with at least one or two additional chemotherapeutic agents. In some embodiments, the at least one additional chemotherapeutic agent is selected from etoposide, carboplatin, cisplatin, and docetaxel. In some embodiments, the at least two additional chemotherapeutic agents are selected from etoposide+carboplatin, ctoposide+cisplatin, and docetaxel+carboplatin.

Certain embodiments include the use of a diagnostic kit for determining therapeutic response to YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, therapy in a human subject with prostate cancer, comprising means for determining (i) AR expression levels. (ii) AR mutation status, and/or (iii) the presence or absence of a neuroendocrine prostate cancer (NEPC) or a small cell carcinoma (SmCC), in a tissue sample from the subject.

In some embodiments, the means for determining (i) comprise reagents for performing a diagnostic assay selected from one or more of immunohistochemistry (IHC) optionally chromogenic or fluorescent IHC, enzyme linked immunosorbent assay (ELISA), or Western blot on a human AR protein or gene, or measuring AR mRNA expression levels. In some embodiments, the means for determining (ii) comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene. In some embodiments, the means for determining (ii) comprise reagents for detecting an activating AR mutation from one or more of one or more of AR gene amplification, one or more activating mutations in the ligand binding domain of AR (optionally selected from H874Y, T877A, T877S, T878A, and F876L), an AR splice variant lacking the ligand binding domain of AR (optionally ARV7 or ARV567), and an E3 ligase MDM2 loss-of-function mutation. In some embodiments, the means for determining (iii) include reagents for performing immunohistochemistry (IHC), optionally for detecting one or more markers selected from synaptophysin, chromogranin A (CgA), neuron-specific enolase (NSE), and CD56.

In some embodiments, the prostate cancer is an Androgen Receptor-Low/Negative (AR^{low/-}) cancer, optionally a castration-resistant prostate cancer (CRPC). In some embodiments, the prostate cancer or CRPC comprises an NEPC, an SmCC, a double negative prostate cancer (DNPC), or an AR low prostate cancer (ARLPC). In some embodiments, the tissue sample is a liquid biopsy optionally a blood sample, a surgical sample, or other biopsy sample obtained from the subject, optionally a biopsy of prostate cancer tissue.

In some embodiments, the diagnostic kit comprises YM155 monobromide, or an analog, derivative, or pharmaceutically acceptable salt thereof. In some embodiments, the diagnostic kit comprises at least one or two additional chemotherapeutic agents. In some embodiments, the at least one additional chemotherapeutic agent is selected from etoposide, carboplatin, cisplatin, and docetaxel. In specific embodiments, at least two additional chemotherapeutic agents are selected from etoposide+carboplatin, etoposide+cisplatin, and docetaxel+carboplatin.

Particular embodiments include a patient care kit, comprising:
(a) means for determining (i) AR expression levels. (ii) AR mutation status, and/or (iii) the presence or absence of a neuroendocrine prostate cancer (NEPC) or a small cell carcinoma (SmCC), in a tissue sample from a human subject with prostate cancer; and
(b) YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof.

In some embodiments, the means for determining (i) comprise reagents for performing a diagnostic assay selected from one or more of immunohistochemistry (IHC) optionally chromogenic or fluorescent IHC, enzyme linked immunosorbent assay (ELISA), or Western blot on a human AR protein or gene, or measuring AR mRNA expression levels. In some embodiments, the means for determining (ii) comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene. In some embodiments, the means for determining (ii) comprise reagents for detecting an activating AR mutation from one or more of one or more of AR gene amplification, one or more activating mutations in the ligand binding domain of AR (optionally selected from H874Y. T877A. T877S. T878A, and F876L), an AR splice variant lacking the ligand binding domain of AR (optionally ARV7 or ARV567), and an E3 ligase MDM2 loss-of-function mutation. In some embodiments, the means for determining (iii) include reagents for performing immunohistochemistry (IHC), optionally for detecting one or more markers selected from synaptophysin, chromogranin A (CgA), neuron-specific enolase (NSE), and CD56.

In some embodiments, the prostate cancer is an Androgen Receptor-Low/Negative (AR^{low/-}) cancer, optionally a castration-resistant prostate cancer (CRPC). In some embodiments, the prostate cancer or CRPC comprises an NEPC, an SmCC, a double negative prostate cancer (DNPC), or an AR low prostate cancer (ARLPC). In some embodiments, the tissue sample is a liquid biopsy optionally a blood sample, a surgical sample, or other biopsy sample obtained from the subject, optionally a biopsy of prostate cancer tissue.

Some embodiments further comprise at least one or two additional chemotherapeutic agents. In some embodiments, the at least one additional chemotherapeutic agent is selected from etoposide, carboplatin, cisplatin, and docetaxel. In some embodiments, the at least two additional chemotherapeutic agents are selected from etoposide+carboplatin, etoposide+cisplatin, and docetaxel+carboplatin.

Also included are pharmaceutical compositions for use in a method of treating an Androgen Receptor-Low/Negative (AR^{low/-}) prostate cancer in a subject in need thereof, comprising YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof. In some embodiments, the AR^{low/-} cancer is a castration-resistant prostate cancer (CRPC). In some embodiments, the prostate cancer or CRPC comprises a neuroendocrine prostate cancer (NEPC), a small cell carcinoma (SmCC), a double negative prostate cancer (DNPC), or an AR low prostate cancer (ARLPC).

In certain embodiments, the pharmaceutical composition further comprises at least one or two additional chemotherapeutic agents. In some embodiments, the at least one additional chemotherapeutic agent is selected from etoposide, carboplatin, cisplatin, and docetaxel. In specific embodiments, the at least two additional chemotherapeutic agents are selected from etoposide+carboplatin, etoposide+cisplatin, and docetaxel+carboplatin.

Certain embodiments relate to the use of a composition in the preparation of a medicament for treating an Androgen Receptor-Low/Negative (AR^{low/-}) prostate cancer, comprising YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof. In some embodiments, the AR^{low/-} cancer is a castration-resistant prostate cancer (CRPC). In some embodiments, the prostate cancer or CRPC comprises a neuroendocrine prostate cancer (NEPC), a small cell carcinoma (SmCC), a double negative prostate cancer (DNPC), or an AR low prostate cancer (ARLPC).

Some embodiments further comprise at least one or two additional chemotherapeutic agents. In some embodiments, the at least one additional chemotherapeutic agent is selected from etoposide, carboplatin, cisplatin, and docetaxel. In some embodiments, the at least two additional chemotherapeutic agents are selected from etoposide+carboplatin, etoposide+cisplatin, and docetaxel+carboplatin.

### BRIEF SUMMARY

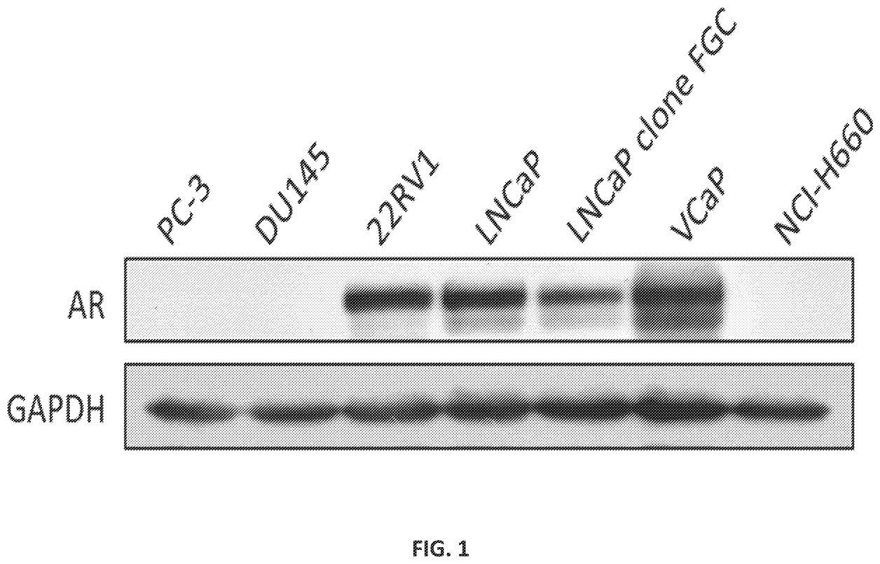

FIG. 1 shows a Western blot analysis of Androgen receptor (AR) expression in metastatic castration-resistant prostate cancer (mCRPC) cells, including PC-3, DU145, 22RV1, LNCaP, LNCaP clone FGC, VcaP, and NCI-H660 cells. GAPDH is shown as a loading control. PC-3, DU145, and NCI-H660 cell lines are AR negative; 22RV1, LNCaP, LNCaP clone FGC, and VCaP cell lines are AR positive.

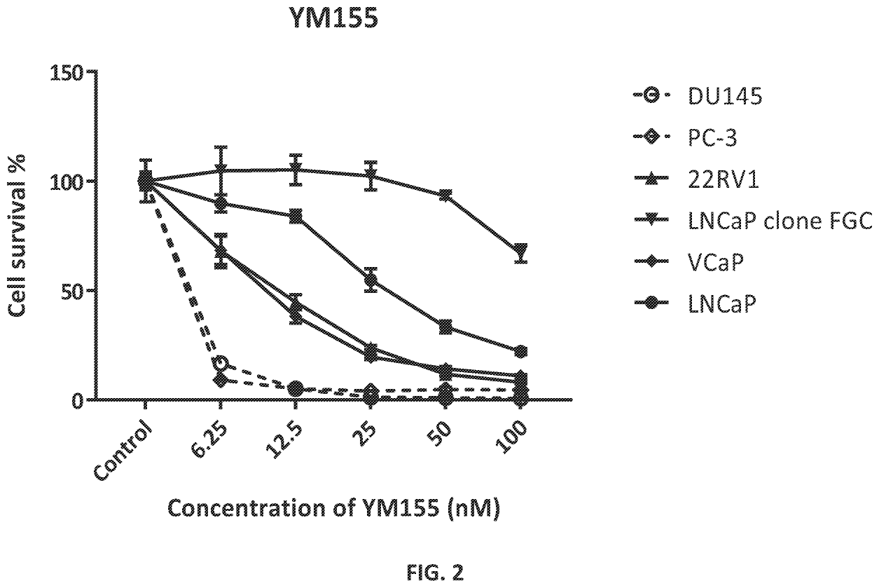

FIG. 2 shows that AR-prostate carcinoma cells are more sensitive to YM155 treatment. The prostate cancer cell lines were treated with Control (DMSO) and YM155 (6.25, 12.5, 25, 50, 100 nM) for 72 hrs. Total cells were compared with cells in control group. Data are presented as mean #SD (n=3).

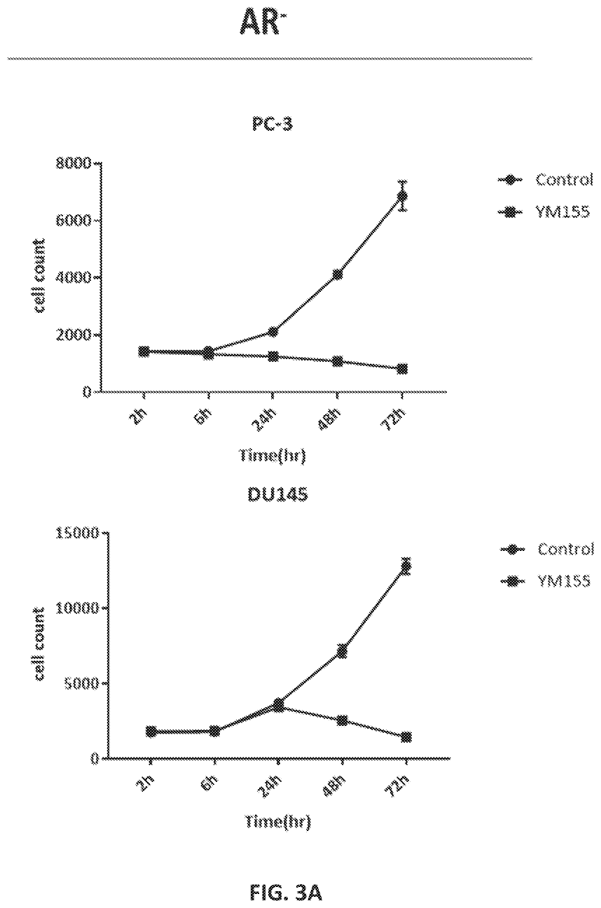

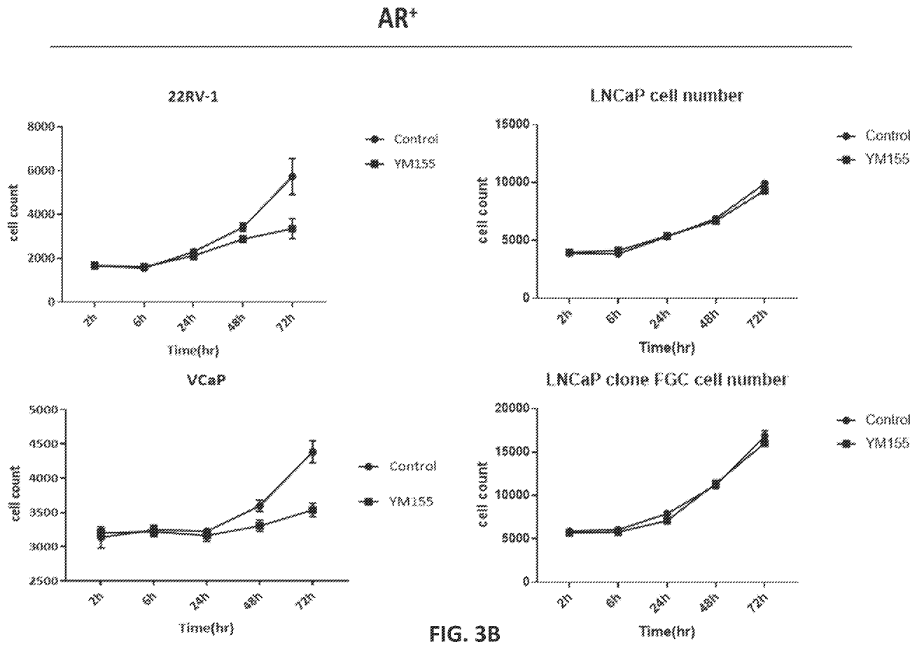

FIGS. 3A-3B show that AR-prostate carcinoma cells are more sensitive to YM155 treatment. AR negative (3A; AR⁻) and AR positive (3B; AR⁺) prostate cancer cells were treated with Control (DMSO) and 10 nM YM155. Total survival cells were analyzed using immunofluorescence at indicated time points. Data are presented as mean±SD (n=3).

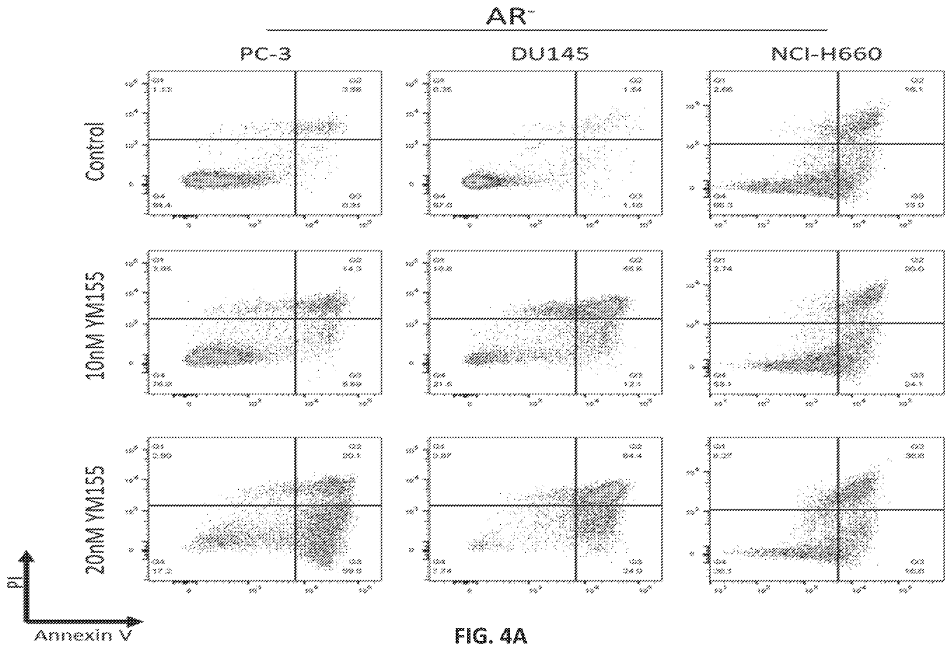

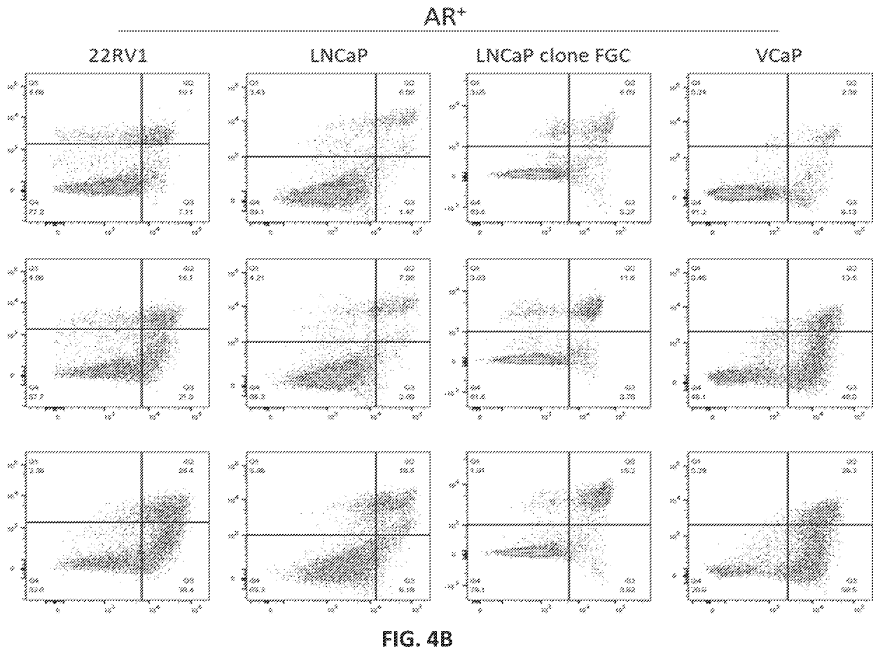

FIGS. 4A-4B show apoptosis analysis with Annexin V/PI in prostate cell lines. The four quadrants Q1, Q2, Q3, and Q4 represent necrosis, late apoptosis, early apoptosis, and live cells, respectively. The prostate cancer cell lines were treated with Control (DMSO) and YM155 (10 nM or 20 nM) for 72 hrs. FIG. 4A shows AR⁻ cells and FIG. 4B shows AR⁺ cells.

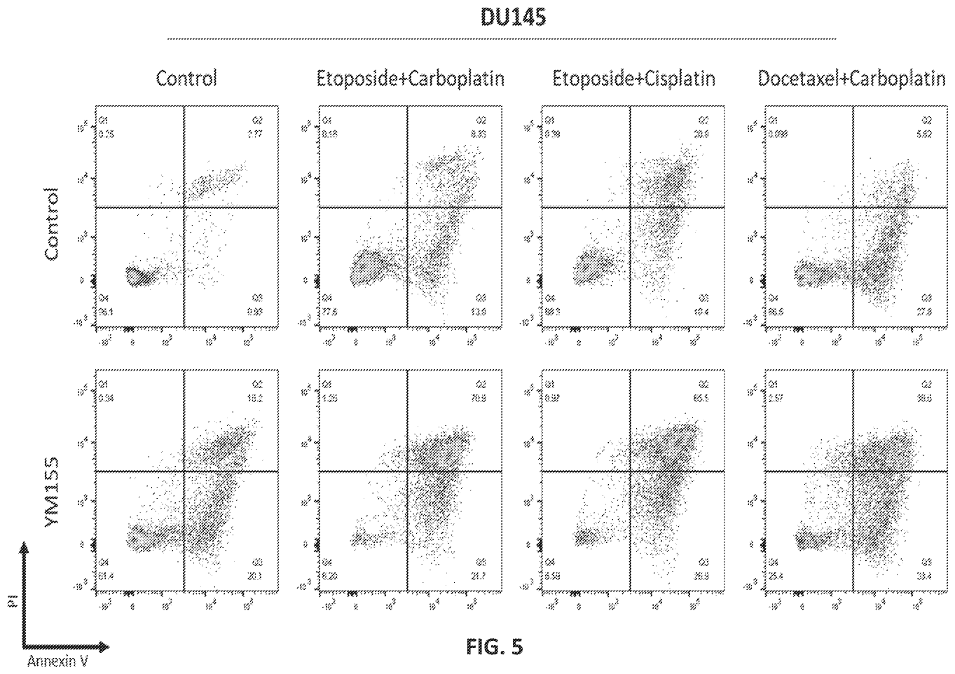

FIG. 5 shows apoptosis analysis with Annexin V/PI in the ART DU145 prostate cancer cell line (CRPC). The four quadrants Q1, Q2, Q3, and Q4 represent necrosis, late apoptosis, early apoptosis, and live cells, respectively. The DU145 cells were treated as indicated: YM155 (10 nM), Etoposide (0.2 μM), Carboplatin (2 μM), Cisplatin (2 μM), and Docetaxel (0.005 μM).

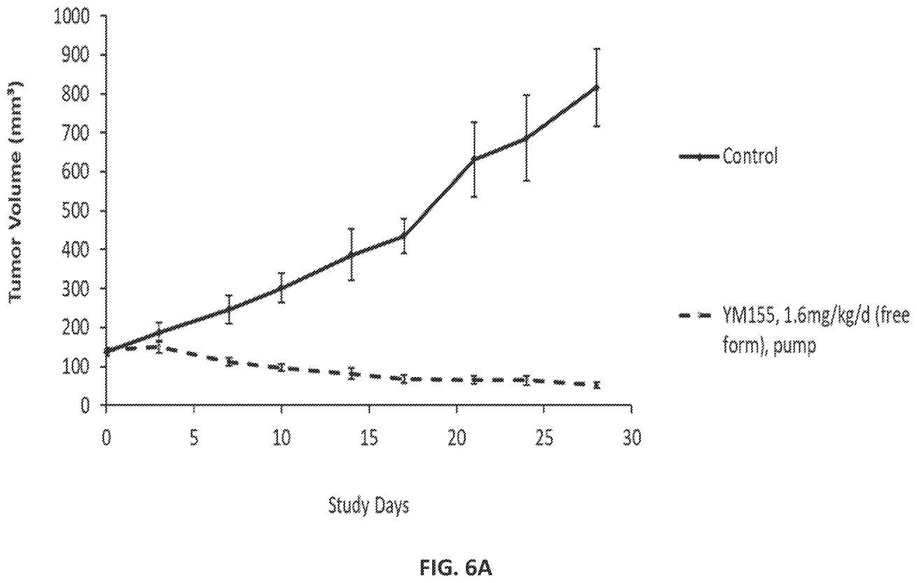

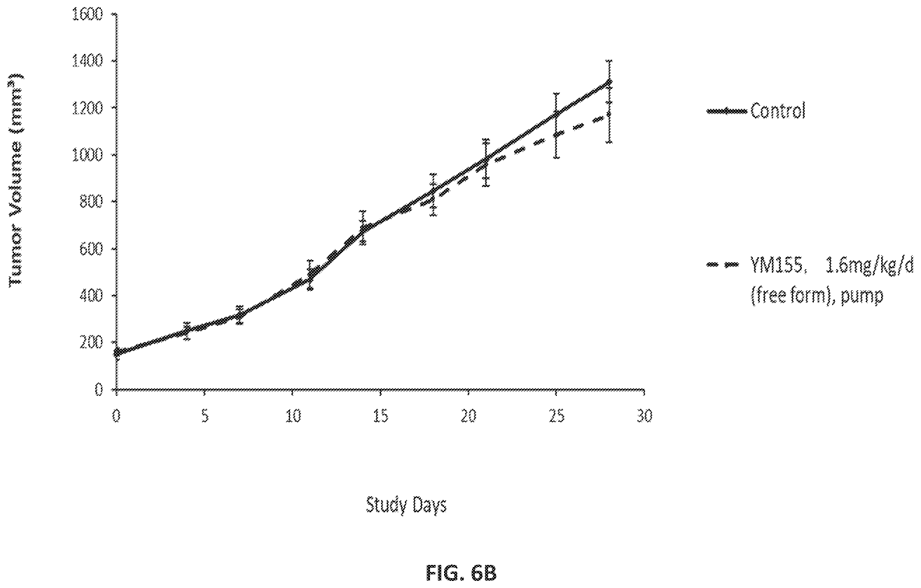

FIGS. 6A-6B show the antitumor activity of YM155 in mice bearing subcutaneously xenografted human prostate tumors. Data are expressed as mean tumor volume±SEM. FIG. 6A shows the effects on PC-3 (AR−) prostate tumors (n=4), and FIG. 6B shows the effects on 22RV1 (AR+) prostate tumors (n=6).

### DETAILED DESCRIPTION

Embodiments of the present disclosure relate to the surprising discovery that prostate cancers comprising an Androgen Receptor (AR^{low/-}) phenotype show significantly higher sensitivity to YM155 monobromide therapy. Thus, AR expression and associated prostate cancer tumor types which have an AR^{low/-} phenotype can be used as biomarkers or companion diagnostics to optimize YM155-related prostate cancer therapies.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

An “antagonist” or “inhibitor” refers to biological structure or chemical agent that interferes with or otherwise reduces the physiological action of another molecule, such as a protein. In some instances, the antagonist or inhibitor specifically binds to the other molecule and/or a functional ligand of the other molecule. In some instances, the antagonist or inhibitor down-regulates the expression of the other molecule. Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

By “about” is meant a quantity, level, value, number, frequency, percentage, dimension, size, amount, weight or length that varies by as much as 20, 15, 10, 9, 8, 7, 6, 5, 4, 3, 2, or 1% to a reference quantity, level, value, number, frequency, percentage, dimension, size, amount, weight, or length.

The term “binding” refers to a direct association between two molecules, due to, for example, covalent, electrostatic, hydrophobic, and ionic and/or hydrogen-bond interactions, including interactions such as salt bridges and water bridges.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “half maximal effective concentration” or “EC₅₀” refers to the concentration of an agent as described herein at which it induces a response halfway between the baseline and maximum after some specified exposure time; the EC₅₀ of a graded dose response curve therefore represents the concentration of a compound at which 50% of its maximal effect is observed. EC so also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

The “half maximal inhibitory concentration” (or “IC₅₀”) is a measure of the potency of an agent in inhibiting a specific biological or biochemical function. This quantitative measure indicates how much of a particular agent (inhibitor) is needed to inhibit a given biological process (or component of a process, i.e, an enzyme, cell, cell receptor or microorganism) by half. The values are typically expressed as molar concentration. The concentration is commonly used as a measure of antagonist drug potency in pharmacological research. In some instances. IC₅₀ represents the concentration of an agent that is required for 50% inhibition in vitro. The IC₅₀ of an agent can be determined by constructing a dose-response curve and examining the effect of different concentrations of the agent on the desired activity, for example, inhibition of tumor cell proliferation, tumor-cell killing.

The “half-life” of an agent refers to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000-fold or more of the amount produced by no composition or a control composition (e.g., the absence of agent or a different agent). An “increased,” “stimulated” or “enhanced” amount may also include an amount that is about or at least about 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, 2000%, 3000%, 4000%, 5000% or more of the amount produced by no composition or a control composition. A “decreased” or “reduced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, or 5000-fold less of the amount produced by no composition or a control composition. A “decreased” or “reduced” amount may also include a 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000%, 2000%, 3000%, 4000%, or 5000% less of the amount produced by no composition or a control composition. Examples of comparisons and “statistically significant” amounts are described herein.

“Prodrug” is meant to indicate a compound that may be converted under physiological conditions or by solvolysis to a biologically active compound described herein, for example, a GSI compound. Thus, the term “prodrug” refers to a metabolic precursor of a compound that is pharmaceutically acceptable. A prodrug may be inactive when administered to a subject in need thereof, but is converted in vivo to an active compound. Prodrugs may be rapidly transformed in vivo to yield the parent compound, for example, by hydrolysis in blood. The prodrug compound often offers advantages of solubility, tissue compatibility or delayed release in a mammalian organism (sec. Bundgard, H., Design of Prodrugs (1985), pp. 7-9, 21-24 (Elsevier, Amsterdam)). A discussion of prodrugs is provided in Higuchi, T., et al., A.C.S. Symposium Series. Vol. 14, and in Bioreversible Carriers in Drug Design, Ed. Edward B. Roche, American Pharmaceutical Association and Pergamon Press, 1987. Examples of prodrugs include, but are not limited to, acetate, formate, and benzoate derivatives of alcohol or amide derivatives of amine functional groups in the compounds of the disclosure and the like.

The term “prodrug” is also meant to include any covalently bonded carriers, which release the active compound in vivo when such prodrug is administered to a subject. Prodrugs of a compound may be prepared by modifying functional groups present in the compound in such a way that the modifications are cleaved, either in routine manipulation or in vivo, to the parent compound. Prodrugs include compounds where a hydroxy, amino, or mercapto group is bonded to any group that, when the prodrug of the compound is administered to a subject, cleaves to form a free hydroxy, free amino, or free mercapto group, respectively.

“Pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, for example, which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, the term “solvate” refers to an aggregate that comprises one or more molecules of a compound described herein with one or more molecules of solvent. The solvent may be water, in which case the solvate may be a hydrate. Alternatively, the solvent may be a biologically-inert organic solvent. Thus, the compounds described herein may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound may merely retain adventitious water or be a mixture of water plus some adventitious solvent.

A “pharmaceutical composition” refers to a formulation of a YM155 compound described herein and a medium generally accepted in the art for the delivery of the biologically active compound to mammals, e.g., humans. Such a medium includes all pharmaceutically acceptable carriers, diluents, and excipients.

The YM155 compounds described herein, or their pharmaceutically-acceptable salts, may contain one or more asymmetric centers and may thus give rise to enantiomers, diastereomers, and other stereoisomeric forms that may be defined, in terms of absolute stereochemistry, as (R)- or(S) or, as (D)- or (L) for amino acids. The present disclosure is meant to include all such possible isomers, as well as their racemic and optically pure forms. Optically active (+) and (−), (R)- and(S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

In certain embodiments, the “purity” of any given agent in a composition may be defined. For instance, certain compositions may comprise an agent that is at least 80%, 85%, 90%, 91%, 92%. 93%, 94%, 95%, 96%, 97%, 98%, 99%, or 100% pure on a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO4). In specific embodiments, solubility is measured at relatively lower pH (e.g., pH 6.0) and relatively higher salt (e.g., 500 mM NaCl and 10 mM NaPO4). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

“Stable compound” and “stable structure” are meant to indicate a compound that is sufficiently robust to survive isolation to a useful degree of purity from a reaction mixture, and formulation into a therapeutic agent.

The term “polynucleotide” and “nucleic acid” includes mRNA, RNA, CRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance. 95%, 96%. 97%, 98%, 99% or greater of some given reference quantity, level, value, number, frequency, percentage, dimension, size, amount, weight, length, or other.

A “stereoisomer” refers to a compound made up of the same atoms bonded by the same bonds but having different three-dimensional structures, which are not interchangeable. The present disclosure includes various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present disclosure includes tautomers of any said compounds.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent needed to elicit the desired biological response following administration.

As used herein. “treatment” of a subject (e.g, a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Certain embodiments include methods for treating an Androgen Receptor-Low/Negative (AR^{low/-}) prostate cancer in a subject in need thereof, comprising administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, thereby treating the AR^{low/-} prostate cancer in the subject in need thereof. The term AR^{low/-} prostate cancer includes a prostate cancer in which AR expression levels in at least some of the tumor cells are absent or significantly reduced relatively to a reference, for example, a healthy control or an AR-dependent prostate cancer. Specific examples of AR^{low/-} prostate cancers include tumors that comprise certain castration-resistant prostate cancer (CRPC) cells (e.g., AR-independent CRPC), neuroendocrine prostate cancers (NEPCs), small cell carcinomas (SmCCs), double negative prostate cancers (DNPCs), and AR low prostate cancers (ARLPCs), including combinations thereof (see, for example, Vellky and Ricke, Neoplasia. 22:566-575, 2020; and Santoni et al., Biochimica et Biophysica Acta. 1846:630-637, 2014).

Some methods include methods of treating an AR^{low/-} prostate cancer include the steps:
(a) determining (i) AR expression levels, (ii) AR mutation status, and/or (iii) the presence or absence of a neuroendocrine prostate cancer (NEPC) or a small cell carcinoma (SmCC), in a tissue sample from the subject; and
(b) administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, to the subject if (i) AR expression levels in the tissue sample are absent (undetectable) or decreased relative to a control or reference, (ii) if the tissue sample lacks an activating AR mutation relative to wild-type AR, and/or (iii) if an NEPC or an SmCC is present in the sample.

Also included are methods for predicting therapeutic response to YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, in a subject with cancer, comprising:
(a) determining (i) AR expression levels, (ii) AR mutation status, and/or (iii) the presence or absence of a neuroendocrine prostate cancer (NEPC) or a small cell carcinoma (SmCC), in a tissue sample from the subject; and
(b) (i) characterizing the subject as responsive to YM155 monobromide therapy if (i) AR expression levels in the tissue sample are absent (undetectable) or decreased relative to a control or reference, (ii) if the tissue sample lacks an activating AR mutation relative to wild-type AR, and/or (iii) if an NEPC or SmCC is present in the tissue sample; or
   (ii) characterizing the subject as non-responsive to YM155 monobromide therapy if (i) AR expression levels in the tissue sample are detectable or normal relative to a control or reference, (ii) if the tissue sample comprises an activating AR mutation, and/or (iii) if an NEPC and an SmCC are absent in the tissue sample,
thereby predicting therapeutic response to YM155 monobromide in the subject with prostate cancer.

Some embodiments include administering YM155 monobromide to the subject if the subject is characterized as responsive to YM155 monobromide therapy. Some instances include administering to the subject a chemotherapeutic agent excluding YM155 monobromide if the subject is characterized as non-responsive to YM155 monobromide therapy.

The androgen receptor (AR), also known as NR3C4 (nuclear receptor subfamily 3, group C, member 4), is a nuclear receptor that is activated by binding any of the androgenic hormones, including testosterone and dihydrotestosterone in the cytoplasm and then translocating into the nucleus. The main function of the androgen receptor is as a DNA-binding transcription factor that regulates gene expression; however, the androgen receptor has other functions as well. Androgen-regulated genes are critical for the development and maintenance of the male sexual phenotype, among other phenotypes.

Prostate cancer initiation and progression is often driven by androgens through binding to the AR (see, Heinlein and Chang, Endocr Rev. 25:276-308, 2004). Initially, this signaling cascade can be successfully targeted with several types of androgen deprivation therapies (ADT). However, some cases (e.g., about 10-20% of cases) progress after androgen deprivation therapy to a more aggressive disease stage, including castration-resistant prostate cancer (CRPC). CRPC can develop by both AR-dependent and AR-independent processes. While the majority of CRPCs remain dependent on AR and androgen signaling, the introduction of more effective AR-targeted therapies can increase the selection for AR^{low/-} subtypes, including CPRCs, which cannot be treated with AR-directed therapies. Moreover, certain prostate tumor subtypes are associated with AR^{low/-} or AR-independent phenotype, including NEPCs, SmCCs, DNPCs, and ARLPCs (Santoni et al., 2014, supra). In some embodiments, the subject has undergone or is undergoing androgen deprivation therapy, and has a prostate cancer that is or has become refractory to androgen deprivation therapy.

“YM155 monobromide” refers to the small molecule [1-(2-Methoxyethyl)-2-methyl-4,9-dioxo-3-(pyrazin-2-ylmethyl)-4,9-dihydro-1H-naphtho [2,3-d] imidazolium bromide], having the molecular formula C₂₀H₁₉N₄O₃·Br, and the CAS Number 781661-94-7 (also sepantronium bromide), and includes pharmaceutically-acceptable salts and acids thereof. Also included are biologically-active or equivalent analogs and/or derivatives of YM155 monobromide, including prodrugs and pharmaceutically-acceptable salts thereof.

As noted above, certain embodiments comprise administering YM155 to the subject if AR levels in the in the tissue sample are absent (i.e., undetectable or substantially undetectable) or decreased relative to that of the control or reference, for example, wherein the control is a healthy tissue or reference derived from a healthy tissue, or an AR-dependent prostate cancer. Certain embodiments comprise administering YM155 to the subject if the AR expression levels in the in the tissue sample are decreased by a statistically significant amount relative to the AR levels of the control or reference. Specific embodiments comprise administering YM155 to the subject if AR expression levels in the in the tissue sample are decreased by about or at least about 1.2, 1.3, 1.4, 1.5, 2, 3, 4, 5, 6, 7, 8, 9, or 10, 50, or 100-fold or more relative to the AR levels of the control or reference.

AR expression levels in a sample of tissue (e.g., cancer tissue) can be determined by any variety of methods. For example, AR protein levels can be determined by immunohistochemistry (IHC) including chromogenic or fluorescent IHC, enzyme linked immunosorbent assay (ELISA), or Western blot on a human AR protein or gene, among other assays. AR mRNA levels can be measured, for example, by RT-PCR, for example, quantitative competitive (QC) RT-PCR, among other techniques known in the art. Certain embodiments thus include the step of determining or detecting or measuring AR levels in a tissue sample from a subject in need thereof. Also included is the step of comparing the AR levels in a tissue sample relative to that of a control or reference.

Some embodiments comprise administering YM155 to the subject if the tissue sample lacks an activating AR mutation relative to wild-type AR. In some instances, an “activating” AR mutation includes at least one alteration (for example, substitutions, insertions, deletions) in the AR protein or in a related ligand/regulatory protein sequence that leads to an increase in AR signaling, for example, relative to wild-type AR. General examples of activating AR mutations include AR gene amplifications, one or more activating mutations in the ligand binding domain of AR (for example, H874Y, T877A, T877S, T878A, F876L), AR splice variants lacking the ligand binding domain of AR (for example, ARV7, ARV567), and E3 ligase MDM2 loss-of-function mutations (see, for example, Vellky and Ricke, 2020, supra).

AR mutation status in a tissue sample can be determined by any variety of methods. For instance, in some embodiments. AR mutation status is determined by DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene. CGH refers to a molecular cytogenetic method for analyzing copy number variations (CNVs) relative to ploidy level in the DNA of a test sample compared to a reference sample, without the need for culturing cells. This technique allows quick and efficient comparisons between two genomic DNA samples arising from two sources, which are most often closely related, because it is suspected that they contain differences in terms of either gains or losses of either whole chromosomes or subchromosomal regions (a portion of a whole chromosome). The technique was originally developed for the evaluation of the differences between the chromosomal complements of solid tumor and normal tissue (see, e.g., Kallioniemi et al., Science. 258 (5083): 818-821, 1992). The use of DNA microarrays in conjunction with CGH techniques has led to the development of a more specific form of array CGH (aCGH), allowing for a locus-by-locus measure of CNV with increased resolution as low as 100 kilobases (see, e.g., Pinkel, Annu Rev Genom Hum Genet. 6:331-354, 2005). In situ hybridization (ISH) and fluorescent in situ hybridization (FISH) refer to a type of hybridization that uses a labeled complementary DNA, RNA or modified nucleic acids strand (i.e., probe) to localize a specific DNA or RNA sequence in a portion or section of tissue (in situ) (see, e.g., Parra & Windle, Nature Genetics. 5: 17-21, 1993; and Gall & Pardue, PNAS USA. 63:378-383, 1969). Thus, the step of determining AR mutation status, for example, to identify activating AR mutations of interest (or their absence), can be performed according to routine techniques in the art. In some instances, the methods and kits described herein employ any one or more of the foregoing techniques and/or comprise reagents for performing the same.

Certain embodiments comprising administering YM155 to the subject if the tissue sample comprises an NEPC or an SmCC, that is, if an NEPC or SmCC or a marker thereof is present in the tissue sample. NEPC refers to an aggressive variant of prostate cancer that commonly arises in later stages of CRPC, but which can also arise de novo in subjects with prostate cancer, and which can be characterized by the downregulation of AR, prostate-specific antigen (PSA), and prostate-specific membrane antigen (PSMA) expression in tumors. SmCC is a high grade tumor characterized by typical nuclear features, such as the lack of prominent nucleoli, nuclear molding, fragility, and crush artifact. NEPC and SmCC are both associated with an AR^{low/-} expression phenotype. The presence or absence of NEPC or SmCC in a tissue sample can be determined according to a variety of techniques in the art, examples of which include cell morphology/histology and/or immunohistochemistry (IHC), for example, via one or more markers such as synaptophysin, chromogranin A (CgA), neuron-specific enolase (NSE), and CD56 (see, for example. Santoni et al., 2014, supra), including the absence of AR expression. In some embodiments, the subject has NEPC if there is at least 50% IHC staining for synaptophysin, CgA, NSE, and/or CD56, including any combination thereof. Certain embodiments include testing for NEPC markers in a liquid biopsy tissue sample. One specific example of a morphological classification of NEPC includes: (1) Usual PCa with NE differentiation: (2) PCa with Paneth cell NE differentiation; (3) Carcinoid tumor; (4) SmCC; (5) Large cell neuroendocrine carcinoma (LCNEC); and (6) Mixed NE carcinoma (SmCC or LCNEC)-acinar adenocarcinoma (see, for example. Epstein et al., Am. J. Surg. Pathol. 38: 756-767, 2014). In some instances, the methods and kits described herein employ any one or more of the foregoing techniques/markers and/or comprise reagents for performing the same.

Examples of a “reference” include a value, amount, sequence, or other characteristic obtained from a database, for example, a “wild-type” AR sequence (see, e.g., ENSG00000169083 and Gene ID: 367 for human AR gene references). A “reference” also includes value, amount, sequence, or other characteristic obtained from a non-cancerous tissue from one or more controls, for example, one or more healthy or non-cancerous control subjects (e.g., a population of healthy or non-cancerous control subjects), or one or more corresponding non-cancerous control tissues from the subject being tested. Typically, a “corresponding” non-cancerous control tissue is obtained from the same type of tissue as the cancer tissue being tested. As with the cancer tissue, the AR levels from a non-cancerous control can be determined by any variety of methods, including, for example, by IHC, for example, chromogenic or fluorescent IHC, ELISA, or Western blot on a human AR protein or gene. Similarly, the AR mutation status from a non-cancerous control can be determined by any variety of methods, including, for example, ISH, FISH, WES, SNP array, NGS, or CGH on a human AR protein or gene.

In some embodiments, the tissue sample is a liquid biopsy (for example, a blood sample), a surgical sample, or other biopsy sample obtained from the subject, including a biopsy of prostate cancer tissue. Certain embodiments include the step of obtaining the tissue sample from the subject, for example, prior to determining AR levels, AR mutation status, and/or the presence or absence of an NEPC or SmCC in the sample.

In some embodiments, the subject is a human subject. In some embodiments, prior to treatment with YM155, the human subject has received at least 1 or 2 lines of systemic therapy for the prostate cancer and has relapsed from the last systemic therapy. Examples of prior systemic therapy include hormonal therapy via surgical or chemical castration (e.g., LHRH agonist), chemotherapy, and/or radiopharmaceutical therapy, including combinations thereof. In specific embodiments, the human subject undergoing YM155 therapy is not undergoing concomitant anti-androgen therapy.

Certain embodiments include combination therapies, for example, administering YM155 to the subject with an AR^{-/low} prostate cancer in combination with at least one or two additional chemotherapeutic agents. In some embodiments, the at least one additional chemotherapeutic agent selected from etoposide, carboplatin, cisplatin, and docetaxel, including combinations thereof. In specific embodiments, the combination of at least two additional chemotherapeutic agents are selected from etoposide+carboplatin, etoposide+cisplatin, and docetaxel+carboplatin.

The methods described herein can be used in the treatment and/or diagnosis of any variety of AR^{-/low} prostate cancers or tumors, including CPRCs, and prostate cancers that comprise NEPCs, SmCCs, double negative prostate cancers (DNPCs), and AR low prostate cancers (ARLPCs), including combinations thereof. NEPCs and SmCCs are described herein. DNPCs stratify samples that are negative for both AR and neuroendocrine markers, while ARLPC indicates cases that lack neuroendocrine markers, but maintain low levels of AR. In some embodiments, the cancer is a primary cancer, that is, a cancer growing at the anatomical site where tumor progression began and yielded a cancerous mass. In some embodiments, the cancer is a secondary or metastatic cancer, that is, a cancer which has spread from the primary site or tissue of origin into one or more different sites or tissues.

In certain embodiments, the methods and compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and compositions described herein increase cancer cell-killing in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, the methods and compositions described herein decrease levels of one or more NEPC markers, such as synaptophysin, CgA, NSE, and CD5, including combinations thereof, by a statistically or clinically-significant amount, for example, by about or at least about 10, 15, 20, 25, 30, 35, 40, 45, 50, 60, 70, 80, 90, 100, 200, 300, 400, 500, 600, 700, 800, 900, 1000, 2000% or more, relative to a control or an earlier timepoint (e.g., before YM155 treatment).

In some embodiments, the methods and compositions described herein increase progression-free survival, overall survival, and/or survival post-progression in the subject in need thereof, for example, by about or at least about 2, 3, 4, 5, 6, 7, 8, 9, 10, 11, 12, 13, 14, 15, 16, 17, 18, 19, 20, 21, 22, 23, or 24 months or more, or by about or at least about 1, 2, 3, 4, 5, 6, 7, 8, 9, or 10 years or more. In certain embodiments, the methods and compositions described are sufficient to result in stable disease. In certain embodiments, the methods and compositions described herein are sufficient to result in clinically relevant reduction in symptoms of a particular disease indication known to the skilled clinician.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, for instance, for the treatment of human disease or testing, the agents described herein are generally incorporated into one or more therapeutic or pharmaceutical compositions prior to administration.

To prepare a therapeutic or pharmaceutical composition, an effective or desired amount of one or more agents is typically mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Certain embodiments include administering a dosage regimen of YM155 via continuous intravenous administration, for example, at about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/m²/day of YM155 by continuous intravenous infusion for about 5, 6, or 7 days. In some instances, the foregoing dosing regimen is repeated every 2, 3, or 4 weeks or so. For certain combination therapies, the YM155 dosage regimen is combined with administration of carboplatin, etoposide, or carboplatin+etoposide. In some instances, the carboplatin is administered over about 30-60) minutes (for example, on day 1 of the YM155 dosage regimen) to achieve an initial target area under the concentration-time curve (AUC) of 4 mg/mL/min (Calvert formula dosing) with standard anti-emetics per practice guidelines, typically wherein the dosage of carboplatin does not exceed about 600 mg/infusion. In some instances, the etoposide is administered intravenously over 60) minutes at about 100 mg/m² (for example, on days 1-3 of the YM155 dosage regimen), optionally following carboplatin administration on day 1 of a carboplatin+etoposide treatment regimen. In certain embodiments, the triple combination of YM155+carboplatin+etoposide increases tumor regression and/or cancer cell-killing in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to the double combination of carboplatin+etoposide.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy. 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline. Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Certain embodiments include the use of a diagnostic kit for determining or predicting a therapeutic response (or responsiveness) to YM155 monobromide [1-(2-Methoxyethyl)-2-methyl-4,9-dioxo-3-(pyrazin-2-ylmethyl)-4,9-dihydro-1H-naphtho [2,3-d] imidazolium bromide] therapy in a subject with prostate cancer, comprising means for determining or measuring (i) AR expression levels. (ii) AR mutation status, and/or (iii) the presence or absence of a neuroendocrine prostate cancer (NEPC) or a small cell carcinoma (SmCC), in a tissue sample from the subject. Also included are patient care kits, comprising: (a) means for determining or measuring (i) AR expression levels, (ii) AR mutation status, and/or (iii) the presence or absence of a neuroendocrine prostate cancer (NEPC) or a small cell carcinoma (SmCC), in a tissue sample from the subject, including cancer tissue and non-cancerous tissue; and (b) YM155 monobromide [1-(2-Methoxyethyl)-2-methyl-4,9-dioxo-3-(pyrazin-2-ylmethyl)-4,9-dihydro-1H-naphtho [2,3-d] imidazolium bromide].

In some embodiments, the means for determining (i) comprise reagents for performing a diagnostic assay selected from one or more of immunohistochemistry (IHC) optionally chromogenic or fluorescent IHC, enzyme linked immunosorbent assay (ELISA), or Western blot on a human AR protein or gene, or measuring AR mRNA expression levels. In some embodiments, the means for determining (ii) comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene. In some embodiments, the means for determining (iii) include reagents for performing immunohistochemistry (IHC), optionally for detecting one or more markers selected from synaptophysin, chromogranin A (CgA), neuron-specific enolase (NSE), and CD56.

Some diagnostic or patient care kits include an AR gene reference obtained from a database, or determined from a non-cancerous tissue from a control or reference. The kits can also include written instructions, for example, on how to determine or measure AR levels. AR mutation status, and/or the presence or absence of a neuroendocrine prostate cancer (NEPC) or a small cell carcinoma (SmCC) in a tissue sample from a subject, and/or from a non-cancerous control.

Certain patient care kits comprise at least one or two additional chemotherapeutic agents, for example, etoposide, carboplatin, cisplatin, and/or docetaxel, including combinations thereof, for instance, etoposide+carboplatin, etoposide+cisplatin, or docetaxel+carboplatin.

In some embodiments, a diagnostic or patient care kit contains separate containers, dividers, or compartments for the composition(s) and informational material(s). For example, the composition(s) or reagents can be contained in a bottle, vial, or syringe, and the informational material(s) can be contained in association with the container. In some embodiments, the separate elements of the kit are contained within a single, undivided container. For example, the composition(s) or reagents are contained in a bottle, vial or syringe that has attached thereto the informational material in the form of a label. In some embodiments, the kit includes a plurality (e.g., a pack) of individual containers, each containing one or more compositions, reagents, and/or unit dosage forms of YM155 monobromide. For example, the kit includes a plurality of syringes, ampules, foil packets, or blister packs, each containing a reagent or a single unit dose of YM155 monobromide. The containers of the kits can be air tight, waterproof (e.g., impermeable to changes in moisture or evaporation), and/or light-tight.

The patient care kit optionally includes a device suitable for administration of the agent(s). e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

In certain aspects, the diagnostic or therapeutic response tests or methods described herein are performed at a diagnostic laboratory, and the results are then provided to the subject, or to a physician or other healthcare provider that plays a role in the subject's healthcare and cancer treatment. Particular embodiments thus include methods for providing the results of the responsiveness test to the subject in need thereof, or to the physician or other healthcare provider. These results or data can be in the form of a hard-copy or paper-copy, or an electronic form, such as a computer-readable medium.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

### Androgen Receptor Status and Responsiveness to YM155 Therapy

Experiments were performed to test the relationship between Androgen Receptor (AR) status and responsiveness of cancer cells to YM155 monotherapy.

Cell Culture. NCI-H660 cells were cultured in RPMI1640 (Hyclone™, USA), supplemented with 5% fetal bovine serum (Gibco, USA), HITES, and Glutamax (Gibco, USA). PC-3 cells were cultured in F-12K (Gibco, USA), supplemented with 10% fetal bovine serum (Gemini, USA) and Glutamax. DU145 cells were cultured in MEM (Gibco, USA), supplemented with 10% fetal bovine serum (Gemini, USA). Glutamax, and Sodium Pyruvate (Gibco, USA). LNcap cells were cultured in RPMI1640 (Hyclone™, USA), supplemented with 10% fetal bovine serum (Gemini, USA) and Glutamax. Vcap cells were cultured in DMEM (Hyclone™, USA), supplemented with 10% fetal bovine serum (Gemini, USA). Glutamax, and Sodium Pyruvate. 22Rv1 and LNcap clone FGC cells were cultured in RPMI1640 (Hyclone™, USA), supplemented with 10% fetal bovine serum (Gibco, USA). Glutamax, and Sodium Pyruvate. All cells were cultured in humidified incubator with 5% CO₂ at 37° C.

NCI-H660 cells were purchased from American Type Culture Collection (USA). LNcap cells were purchased from Cell Resource Center, IBMS, CAMS/PUMC (Beijing, China), PC-3, DU145, 22Rv1, Vcap, and LNcap FGC cells were purchased from Cell Bank, Shanghai Institutes for Biological Sciences, Chinese Academy of Sciences (Shanghai, China).

Western Blot. NCI-H660, PC-3, DU145, 22Rv1, LNCaP, VcaP, and LNCaP clone FGC cells were harvested and centrifuged at 500 g for 5 minutes to obtain cell pellets. The pellets were lysed in lysis buffer (Beyotime, China) which included a protease inhibitor cocktail (Beyotime, China) and a phosphatase inhibitor cocktail (Beyotime, China). Cells were incubated on ice for 30 minutes and then centrifuged at 4° C., 12000 rpm for 10 minutes to obtain the supernatant as cell lysate. The concentrations of protein in cell lysate were determined by Micro BCA™ Protein Assay Kit (ThermoFisher, USA). 4× SDS-PAGE Sample Loading Buffer (Beyotime, China) was added into cell lysate contained 30 ∪g of total protein and, after boiling, the mixture was electrophoresed in polyacrylamide gel. After electrophoresis, proteins on the gel were transferred to a PVDF membrane, and the membrane was cut at position close to the molecular weights of proteins whose expression was examined (Androgen Receptor (CST, 68492S), GAPDH (ZSGB-BIO, TA-08)).

As shown in FIG. 1, PC-3, DU145, and NCI-H660 cell lines are AR negative, and 22RV1, LNCaP, LNCaP clone FGC, and VCaP cell lines are AR positive.

Cell Treatment and proliferation assay. PC-3 and DU145 cells were seeded in 96-well plates (Corning, USA) at 2000 cells/well; LNCaP, VcaP, and LNCaP clone FGC cells were seeded at 5000 cells/well; and 22Rv1 cells were seeded at 3000 cells/well. Cells were treated with 10 nM YM155 or 0.1% DMSO (VWR, USA) for different times (2, 6, 24, 48, and 72 hrs). DU145 cells were also treated with YM155 (10 nM) in further combination with etoposide (0.2 ∪M)+carboplatin (2 ∪M), etoposide (0.2 ∪M)+cisplatin (2 μM), or docetaxel (0.005 μM)+carboplatin (2 μM). At indicated times, cells were fixed with Formaldehyde (final concentration at 4 or 5%; Thermo, USA) for 30-40 minutes at room temperature. The cells were washed three times with PBS (Cellmax, China), then blocked and permeabilized with 3% BSA (Sigma, USA) in 0.5% Triton X-100 (Sigma, USA) for 2 hrs at room temperature. After discarding the supernatant, the cells were incubated with DAPI (Beyotime, China) for 10 minutes at room temperature, then washed again three times with PBS. The cells were kept in PBS and protected from light throughout experiments. The treated cells were scanned for image acquisition with CellInsight™ CX5 High-Content Screening (HSC) Platform (Thermo Fisher), equipped with filters for DAPI (Ex: 386 nm).

PC-3, DU145, 22Rv1, LNCaP, VcaP, and LNCaP clone FGC cells were seeded in 96-well plates at 2000 cells/well. Cells were treated with YM155 (100, 50, 25, 12.5, 6.25 nM) or 0.1% DMSO for 72 hours. After incubation with EdU (Thermo, USA), cells were fixed with Formaldehyde (final concentration at 4%) for 30 min at room temperature. The cells were washed three times with PBS, then permeabilized with 0.5% Triton X-100 in PBS overnight at 4° C. After discarding the supernatant, the cells were incubated with Hoechst 33342 (Invitrogen, USA) for 1 hr at room temperature, then washed again three times with PBS. Cells were incubated with staining mix (Beijing Percans Oncology Medical Research Co., Ltd., RUO-00401 #150T) for 30 min at room temperature, and then washed three times with PBS. The cells were kept in PBS and protected from light throughout experiments.

High content imaging analysis. The treated cells were scanned for image acquisition with CellInsight™ CX5 HSC Platform, equipped with filters for Hoechst33342 (Ex: 386 nm) and EdU (Ex: 560 nm). The total cell count and EdU-positive cell count were analysis by measuring the signal intensity in the nuclear region.

As shown in FIG. 2 and FIGS. 3A-3B. AR⁻ prostate carcinoma cells are more sensitive to YM155 treatment than AR+prostate carcinoma cells

Flow Cytometry. NCI-H660, PC-3, DU145, 22Rv1, LNCaP, VcaP, and LNCaP clone FGC cells were treated with YM155 (10 nM, 20 nM) for 72 hours (0.1% DMSO was added as control). Cells were harvest and centrifuged at 500 g for 5 minutes to obtain cell pellets, then washed the cells once with PBS. Cells were stained with propidium iodide (PI) and Annexin V using apoptosis detection kits (Thermo, USA) and analyzed on a BD FACS Analyzer (LSRFortessa, USA). Data were analyzed using FlowJo software. The results are shown in FIGS. 4A (AR⁻) and 4B (AR⁺). The results for the combination treatments are shown in FIG. 5.

Animal Models. Five or six-week old male nude mice were purchased from SPF Biotechnology Co., Ltd. (Beijing, China). The prostate tumor cell suspensions (PC-3 or 22RV1) were grafted subcutaneously into the flank of nude mice at a concentration of 3×10⁶ cells/mouse. Eight mice whose tumor volumes length×width²×0.5 (length×width²×0.5) reached approximately 100 mm³ were divided into groups. For 21-days subcutaneous (s.c.) continuous infusion, an osmotic pump containing YM155 was implanted in the dorsum of each animal. Body weight and tumor diameter were measured every 3-4 days. The results are shown in FIG. 6A (AR⁻) and 6B (AR⁺). Anti-tumor activities are expressed as percent inhibition of tumor growth. The percent inhibition of tumor growth on day 21/28 was calculated using following formula: Mean % Inhibition=(mean (Control)−mean (YM155))/mean (Control)*100%. The treatment was started at day 0; vertical bars, SEM. In these experiments, YM155 showed 89.62% and 93.63% inhibition of PC-3 (AR⁻) tumor growth relative to control at day 21 and 28, respectively.***: P<0.001. In contrast, YM155 showed no significant difference (n.s.) in activity towards 22RV1 (AR⁺) cells relative to control.

1. A method for treating an Androgen Receptor-Low/Negative (AR^{low/-}) prostate cancer in a subject in need thereof, comprising
administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof,
thereby treating the AR^{low/-} prostate cancer in the subject in need thereof.

2. The method of claim 1, comprising,
(a) determining (i) AR expression levels, (ii) AR mutation status, and/or (iii) the presence or absence of a neuroendocrine prostate cancer (NEPC) or a small cell carcinoma (SmCC), in a tissue sample from the subject; and
(b) administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, to the subject if (i) AR expression levels in the tissue sample are absent (undetectable) or decreased relative to a control or reference, (ii) if the tissue sample lacks an activating AR mutation relative to wild-type AR, and/or (iii) if an NEPC or an SmCC is present in the sample.

3. A method for predicting therapeutic response to YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, in a subject with prostate cancer, comprising
(a) determining (i) AR expression levels, (ii) AR mutation status, and/or (iii) the presence or absence of a neuroendocrine prostate cancer (NEPC) or a small cell carcinoma (SmCC), in a tissue sample from the subject; and
(b) (i) characterizing the subject as responsive to YM155 monobromide therapy if (i) AR expression levels in the tissue sample are absent (undetectable) or decreased relative to a control or reference, (ii) if the tissue sample lacks an activating AR mutation relative to wild-type AR, and/or (iii) if an NEPC or SmCC is present in the tissue sample; or
(ii) characterizing the subject as non-responsive to YM155 monobromide therapy if (i) AR expression levels in the tissue sample are detectable or normal relative to a control or reference, (ii) if the tissue sample comprises an activating AR mutation, and/or (iii) if an NEPC and an SmCC are absent in the tissue sample,
thereby predicting therapeutic response to YM155 monobromide in the subject with prostate cancer.

4. The method of claim 3, comprising administering the YM155 monobromide to the subject if the subject is characterized as responsive to YM155 monobromide therapy.

5. The method of any one of claims 1-4, comprising determining AR expression levels in the tissue sample by immunohistochemistry (IHC) optionally chromogenic or fluorescent IHC, enzyme linked immunosorbent assay (ELISA), or Western blot on a human AR protein or gene, or by measuring AR mRNA expression levels.

6. The method of any one of claims 1-5, comprising administering the YM155 to the subject if AR levels in the tissue sample are undetectable or decreased by about or at least about 2, 3, 4, 5, 6, 7, 8, 9, or 10, 50, or 100-fold or more relative to the AR levels of the control or reference, optionally wherein the control is a healthy tissue.

7. The method of any one of claims 1-6, comprising determining AR mutation status in the tissue sample by DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene.

8. The method of any one of claims 1-7, wherein the activating AR mutation is selected from one or more of one or more of AR gene amplification, one or more activating mutations in the ligand binding domain of AR (optionally selected from H874Y, T877A, T877S, T878A, and F876L), an AR splice variant lacking the ligand binding domain of AR (optionally ARV7 or ARV567), and an E3 ligase MDM2 loss-of-function mutation.

9. The method of any one of claims 1-8, wherein the AR^{low/-} prostate cancer is a castration-resistant prostate cancer (CRPC).

10. The method of claim 9, wherein the prostate cancer or CRPC comprises an NEPC, an SmCC, a double negative prostate cancer (DNPC), or an AR low prostate cancer (ARLPC).

11. The method of claim 10, wherein (iii) comprises determining the presence of the NEPC or SmCC in the tissue sample by cell morphology/histology, the absence of AR expression, and/or by immunohistochemistry (IHC), optionally via one or more markers selected from synaptophysin, chromogranin A (CgA), neuron-specific enolase (NSE), and CD56.

12. The method of any one of claims 1-11, comprising obtaining the tissue sample from the subject.

13. The method of any one of claims 1-12, wherein the tissue sample is a liquid biopsy optionally a blood sample, a surgical sample, or other biopsy sample obtained from the subject, optionally a biopsy of prostate cancer tissue.

14. The method of any one of claims 1-13, wherein the subject is a human subject, optionally wherein the human subject has received at least 1 or 2 lines of systemic therapy for the prostate cancer and has relapsed from the last systemic therapy, optionally hormonal therapy via surgical or chemical castration (LHRH agonist), chemotherapy, or radiopharmaceutical therapy.

15. The method of any one of claims 1-14, comprising administering the YM155 in combination with at least one or two additional chemotherapeutic agents.

16. The method of claim 15, wherein the at least one additional chemotherapeutic agent is selected from etoposide, carboplatin, cisplatin, and docetaxel.

17. The method of claim 15, wherein the at least two additional chemotherapeutic agents are selected from etoposide+carboplatin, etoposide+cisplatin, and docetaxel+carboplatin.

18. Use of a diagnostic kit for determining therapeutic response to YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, therapy in a human subject with prostate cancer, comprising means for determining (i) AR expression levels, (ii) AR mutation status, and/or (iii) the presence or absence of a neuroendocrine prostate cancer (NEPC) or a small cell carcinoma (SmCC), in a tissue sample from the subject.

19. The use claim 18, wherein the means for determining (i) comprise reagents for performing a diagnostic assay selected from one or more of immunohistochemistry (IHC) optionally chromogenic or fluorescent IHC, enzyme linked immunosorbent assay (ELISA), or Western blot on a human AR protein or gene, or measuring AR mRNA expression levels.

20. The use of claim 18 or 19, wherein the means for determining (ii) comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene.

21. The use of claim 20, wherein the means for determining (ii) comprise reagents for detecting an activating AR mutation from one or more of one or more of AR gene amplification, one or more activating mutations in the ligand binding domain of AR (optionally selected from H874Y, T877A, T877S, T878A, and F876L), an AR splice variant lacking the ligand binding domain of AR (optionally ARV7 or ARV567), and an E3 ligase MDM2 loss-of-function mutation.

22. The use of any one of claims 18-21, wherein the means for determining (iii) include reagents for performing immunohistochemistry (IHC), optionally for detecting one or more markers selected from synaptophysin, chromogranin A (CgA), neuron-specific enolase (NSE), and CD56.

23. The use of any one of claims 18-22, wherein the prostate cancer is an Androgen Receptor-Low/Negative (AR^{low/-}) cancer, optionally a castration-resistant prostate cancer (CRPC).

24. The use of claim 23, wherein the prostate cancer or CRPC comprises an NEPC, an SmCC, a double negative prostate cancer (DNPC), or an AR low prostate cancer (ARLPC).

25. The use of any one of claims 18-24, wherein the tissue sample is a liquid biopsy optionally a blood sample, a surgical sample, or other biopsy sample obtained from the subject, optionally a biopsy of prostate cancer tissue.

26. The use of any one of claims 18-25, wherein the diagnostic kit comprises YM155 monobromide, or an analog, derivative, or pharmaceutically acceptable salt thereof.

27. The use of claim 26, wherein the diagnostic kit comprises at least one or two additional chemotherapeutic agents.

28. The use of claim 27, wherein the at least one additional chemotherapeutic agent is selected from etoposide, carboplatin, cisplatin, and docetaxel.

29. The use of claim 27, wherein the at least two additional chemotherapeutic agents are selected from etoposide+carboplatin, etoposide+cisplatin, and docetaxel+carboplatin.

30. A patient care kit, comprising:
(a) means for determining (i) AR expression levels, (ii) AR mutation status, and/or (iii) the presence or absence of a neuroendocrine prostate cancer (NEPC) or a small cell carcinoma (SmCC), in a tissue sample from a human subject with prostate cancer; and
(b) YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof.

31. The patient care kit of claim 30, wherein the means for determining (i) comprise reagents for performing a diagnostic assay selected from one or more of immunohistochemistry (IHC) optionally chromogenic or fluorescent IHC, enzyme linked immunosorbent assay (ELISA), or Western blot on a human AR protein or gene, or measuring AR mRNA expression levels.

32. The patient care kit of claim 30 or 31, wherein the means for determining (ii) comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human AR protein or gene.

33. The patient care kit of claim 32, wherein the means for determining (ii) comprise reagents for detecting an activating AR mutation from one or more of one or more of AR gene amplification, one or more activating mutations in the ligand binding domain of AR (optionally selected from H874Y, T877A, T877S, T878A, and F876L), an AR splice variant lacking the ligand binding domain of AR (optionally ARV7 or ARV567), and an E3 ligase MDM2 loss-of-function mutation.

34. The patient care kit of any one of claims 30-33, wherein the means for determining (iii) include reagents for performing immunohistochemistry (IHC), optionally for detecting one or more markers selected from synaptophysin, chromogranin A (CgA), neuron-specific enolase (NSE), and CD56.

35. The patient care kit of any one of claims 30-34, wherein the prostate cancer is an Androgen Receptor-Low/Negative (AR^{low/-}) cancer, optionally a castration-resistant prostate cancer (CRPC).

36. The patient care kit of claim 35, wherein the prostate cancer or CRPC comprises an NEPC, an SmCC, a double negative prostate cancer (DNPC), or an AR low prostate cancer (ARLPC).

37. The patient care kit of any one of claims 30-36, wherein the tissue sample is a liquid biopsy optionally a blood sample, a surgical sample, or other biopsy sample obtained from the subject, optionally a biopsy of prostate cancer tissue.

38. The patient care kit of any one of claims 30-37, further comprising at least one or two additional chemotherapeutic agents.

39. The patient care kit of claim 38, wherein the at least one additional chemotherapeutic agent is selected from etoposide, carboplatin, cisplatin, and docetaxel.

40. The patient care kit of claim 38, wherein the at least two additional chemotherapeutic agents are selected from etoposide+carboplatin, etoposide+cisplatin, and docetaxel+carboplatin.

41. A pharmaceutical composition for use in a method of treating an Androgen Receptor-Low/Negative (AR^{low/-}) prostate cancer in a subject in need thereof, comprising YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof.

42. The pharmaceutical composition for use according to 41, wherein the AR^{low/-} cancer is a castration-resistant prostate cancer (CRPC).

43. The pharmaceutical composition for use according to 41 or 42, wherein the prostate cancer or CRPC comprises a neuroendocrine prostate cancer (NEPC), a small cell carcinoma (SmCC), a double negative prostate cancer (DNPC), or an AR low prostate cancer (ARLPC).

44. The pharmaceutical composition for use according to any one of claims 41-43, further comprising at least one or two additional chemotherapeutic agents.

45. The pharmaceutical composition for use according to claim 44, wherein the at least one additional chemotherapeutic agent is selected from etoposide, carboplatin, cisplatin, and docetaxel.

46. The pharmaceutical composition for use according to claim 44, wherein the at least two additional chemotherapeutic agents are selected from etoposide+carboplatin, etoposide+cisplatin, and docetaxel+carboplatin.

47. Use of a composition in the preparation of a medicament for treating an Androgen Receptor-Low/Negative (AR^{low/-}) prostate cancer, comprising YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof.

48. The use of claim 47, wherein the AR^{low/-} cancer is a castration-resistant prostate cancer (CRPC).

49. The use of claim 47 or 48, wherein the prostate cancer or CRPC comprises a neuroendocrine prostate cancer (NEPC), a small cell carcinoma (SmCC), a double negative prostate cancer (DNPC), or an AR low prostate cancer (ARLPC).

50. The use according to any one of claims 47-49, further comprising at least one or two additional chemotherapeutic agents.

51. The use according to claim 50, wherein the at least one additional chemotherapeutic agent is selected from etoposide, carboplatin, cisplatin, and docetaxel.

52. The use according to claim 50, wherein the at least two additional chemotherapeutic agents are selected from etoposide+carboplatin, etoposide+cisplatin, and docetaxel+carboplatin.
